(19) United States
(12) Patent Application Publication
(10) Pub. No.: US 2020/0043572 A1
(43) Pub. Date: Feb. 6, 2020

(54) BIOLOGICAL DATABASE INCORPORATING CONTENT-PROVIDER PAYMENT MECHANISM

(71) Applicant: LIFE TECHNOLOGIES CORPORATION, Carlsbad, CA (US)

(72) Inventor: Paul Grossman, Del Mar, CA (US)

(21) Appl. No.: 16/544,320

(22) Filed: Aug. 19, 2019

Related U.S. Application Data
(63) Continuation of application No. 15/240,458, filed on Aug. 18, 2016, now abandoned.
Application No. 15/240,458 is a division of application No. 13/369,663, filed on Feb. 9, 2012, now abandoned.
(60) Provisional application No. 61/441,400, filed on Feb. 10, 2011.

(51) Int. Cl. G16B 50/00; G06Q 30/06; G06Q 30/02

(52) U.S. Cl. CPC G16B 50/00; G06Q 30/0283; G06Q 30/06

(57) ABSTRACT

Systems and methods are used to provide an aggregated genomic information database and compensate contributors. Genomic data and contributor data are received from a contributor client device and stored in an electronic database using a server computer. A linkage between the genomic data and other genomic data stored in the electronic database is determined and the linkage is stored in the electronic database using the server computer. A request for the genomic data from a user client device is received, the genomic data and the linkage are retrieved from the electronic database, and the genomic data and the linkage are sent to the user client device using the server computer. A compensation amount value for the genomic data is calculated and sent to the contributor client device using the server computer.

### RELATED APPLICATIONS

This application is a continuation of U.S. application Ser. No. 15/240,458 filed Aug. 18, 2016, which is a divisional of U.S. application Ser. No. 13/369,663 filed Feb. 9, 2012, which claims priority to U.S. application No. 61/441,400 filed Feb. 10, 2011, which disclosures are herein incorporated by reference in their entirety.

### FIELD

The present disclosure relates to a biological database incorporating content-provider payment mechanism

### INTRODUCTION

Biological information is widely available. Nucleic acid and protein sequences can be retrieved from, compared with, or added to large publicly available databases. Scientific papers can be searched for information that links therapeutic information or biological significance to genomic or genetic information. In addition, increasingly less expensive and fast laboratory instruments can produce genomic and genetic information in forms that are easily understood and exchanged.

Unfortunately, the wide availability of biological information has not significantly increased the speed at which diseases have been cured or pharmaceuticals have been developed. Part of the problem has been the inability to successfully aggregate all the information known about each piece of biological information in one place.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a block diagram that illustrates a computer system, upon which embodiments of the present teachings may be implemented.

FIG. 2 is a schematic diagram showing a system for providing an aggregated genomic information database, in accordance with various embodiments.

FIG. 3 is a schematic diagram showing a system for aggregating genomic information, in accordance with various embodiments.

FIG. 4 is a flowchart showing a method for generating a genomic database, in accordance with various embodiments.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DESCRIPTION OF VARIOUS EMBODIMENTS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the present teachings.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

As utilized in accordance with the embodiments provided herein, the following terms, unless otherwise indicated, shall be understood to have the following meanings:

As used herein, “a” or “an” means “at least one” or “one or more”. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

### Computer-Implemented System

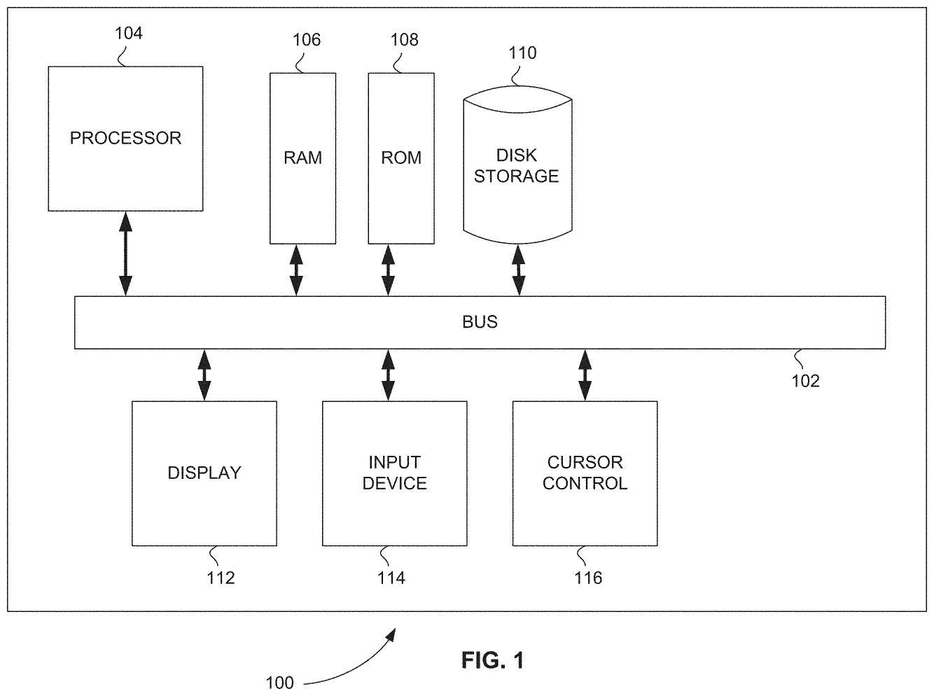

FIG. 1 is a block diagram that illustrates a computer system 100, upon which embodiments of the present teachings may be implemented. Computer system 100 can include a bus 102 or other communication mechanism for communicating information, and a processor 104 coupled with bus 102 for processing information. Computer system 100 can also include a memory 106, which can be a random access memory (RAM) or other dynamic storage device, coupled to bus 102 for determining base calls, and instructions to be executed by processor 104. Memory 106 also may be used for storing temporary variables or other intermediate information during execution of instructions to be executed by processor 104. Computer system 100 can further include a read only memory (ROM) 108 or other static storage device coupled to bus 102 for storing static information and instructions for processor 104. A storage device 110, such as a magnetic disk or optical disk, can be provided and coupled to bus 102 for storing information and instructions.

Computer system 100 may be coupled via bus 102 to a display 112, such as a cathode ray tube (CRT) or liquid crystal display (LCD), for displaying information to a computer user. An input device 114, including alphanumeric and other keys, can be coupled to bus 102 for communicating information and command selections to processor 104. Another type of user input device can be cursor control 116, such as a mouse, a trackball or cursor direction keys for communicating direction information and command selections to processor 104 and for controlling cursor movement on display 112. This input device typically can have two degrees of freedom in two axes, a first axis (i.e., x) and a second axis (i.e., y), that allows the device to specify positions in a plane. In various embodiments, functionality of display 112, input device 114, and cursor control 116 can be combined, such as in a touch screen display.

A computer system 100 can perform the present teachings. Consistent with certain implementations of the present teachings, results can be provided by computer system 100 in response to processor 104 executing one or more sequences of one or more instructions contained in memory 106. Such instructions may be read into memory 106 from another computer-readable medium, such as storage device 110. Execution of the sequences of instructions contained in memory 106 can cause processor 104 to perform the process described herein. Alternatively hard-wired circuitry may be used in place of or in combination with software instructions to implement the present teachings. Thus, implementations of the present teachings are not limited to any specific combination of hardware circuitry and software.

The term “computer-readable medium” as used herein refers to any media that participates in providing instructions to processor 104 for execution. Such a medium may take many forms, including but not limited to, non-volatile media, volatile media, and transmission media. Non-volatile media includes, for example, optical or magnetic disks, such as storage device 110. Volatile media includes dynamic memory, such as memory 106. Transmission media includes coaxial cables, copper wire, and fiber optics, including the wires that comprise bus 102.

Common forms of non-transitory computer-readable media include, for example, a floppy disk, a flexible disk, hard disk, magnetic tape, or any other magnetic medium, a CD-ROM, any other optical medium, punch cards, papertape, any other physical medium with patterns of holes, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 104 for execution. For example, the instructions may initially be carried on the magnetic disk of a remote computer. The remote computer can load the instructions into its dynamic memory and send the instructions over a network, such as a wired network or a wireless network. A network interface controller of computer system 100 can receive the data from the network and place the data on bus 102. Bus 102 can carry the data to memory 106, from which processor 104 can retrieve and can execute the instructions. The instructions received by memory 106 may optionally be stored on storage device 110 either before or after execution by processor 104.

In accordance with various embodiments, instructions configured to be executed by a processor to perform a method can be stored on a non-transitory and tangible computer-readable medium. The computer-readable medium can be a device that stores digital information. For example, a computer-readable medium can include a compact disc read-only memory (CD-ROM) as is known in the art for storing software. The computer-readable medium can be accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software but the present teachings may be implemented as a combination of hardware and software or in hardware alone. The present teachings may be implemented with both object-oriented and non-object-oriented programming systems.

Systems and Methods of Data Processing

As described above, the wide availability of biological information has not significantly increased the speed at which diseases have been cured or pharmaceuticals have been developed. Part of the problem has been the inability to successfully aggregate all the information known about each piece of biological information in one place. Biological information can include sequence information, such as DNA sequences, RNA sequences, amino acid sequences, and the like, variations in sequence information, phenotype information, relationships between sequence information and medical conditions or treatment outcomes, etc. Herein, in various embodiments, reference is made to genetic or genomic sequence information by way of example. One of skill in the art would understand that, in the context of the disclosed embodiments, other types of biological information can be readily substituted for the genetic or genomic sequence information.

In various embodiments, content-providers, or contributors can be given an incentive to contribute information to a single database in order to aggregate as much known information as possible about each genetic or genomic sequence in that database. This can allow the aggregation of genomic content having medical significance into one integrated data set. The incentive may include a payment that is recorded and made to each contributor for each contribution of information. The incentive may include a credit towards use of a service, preferred access to a service, early access to a service, a reduced price of the service, or any combination thereof.

The information contributed to the database can include a medically relevant genomic variation. The information can also include a relationship between the variation and a medical condition of a patient. The variation-medical relationship can be established through published data or proprietary studies, for example. The information can also include information about a level of gene expression (e.g., up-regulated or down-regulated) associated with a particular medical condition or particular set of circumstances, for example. It can also include information about the presence of a disease or condition, the propensity to develop a certain disease or condition, and/or an increased sensitivity to treatment or a resistance to a treatment.

The payment can be made to each contributor for each contribution of information may be, for example, proportional to the value or reliability of the information provided. It may also be a flat rate. The value of a contribution can be established based on a number of factors including, but not limited to, the number of times a contribution is accessed by users, the level of reliability or validation of the contribution, whether the contribution is public or proprietary information, or the scientific of informational depth of the contribution.

The information contributed to the database can be binned into one or more categories based on its value. For example the database can include three categories: low, medium, and high. The low category can include information that is publicly available and has a low level of validation. The medium category can include information that is proprietary and has a low level of validation. Finally, the high category can include information that is proprietary and has a high level of validation. The information may also be binned in other ways that distinguish more or less finely a plurality of discrete groups by value and/or validation.

Contributors can be given additional incentives to contribute to the database. For example, contributors can be given preferred access, early access, or a reduced price for access to the database. Users, whether they also contribute to the database or not, may be given partial or complete access to the information binned in a plurality of discrete groups according to value and/or validation. For example, users may be given access to a subset of information having a lesser value and/or validation at a minimal or no cost, but they may have to pay additional access fees to access information having a higher value and/or validation. The additional access fees may be structured to give access in block to any element of information included in one of the plurality of discrete groups distinguishing information by value and/or validation. Alternatively, the users may have to pay additional access fees for certain specific elements of information that are deemed to have a very high value and/or validation. Other fee arrangements may be negotiated for particular users according to their particular situations and usage.

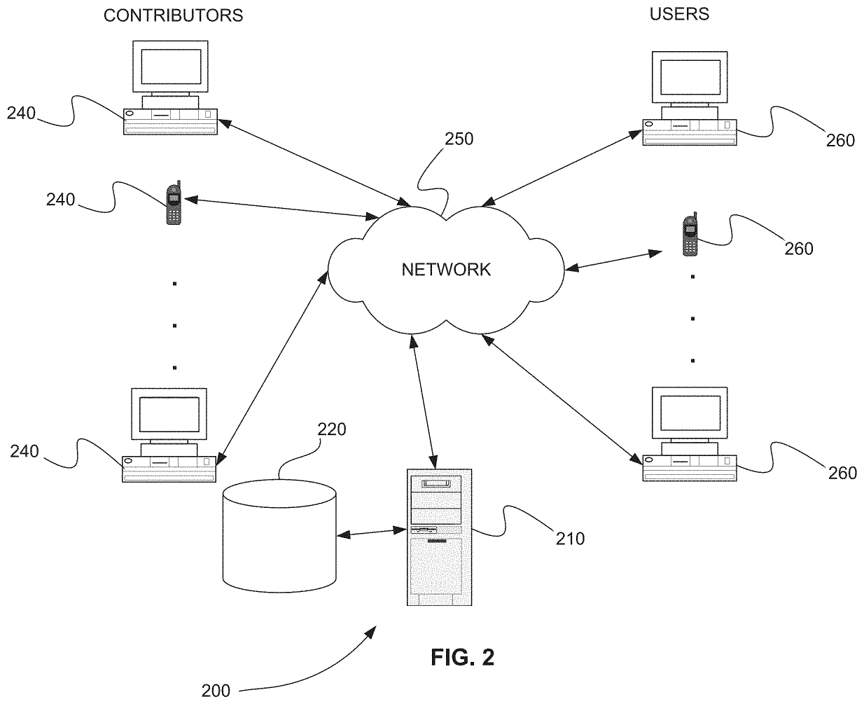

FIG. 2 is a schematic diagram showing a system 200 for aggregating and valuing genomics content, in accordance with various embodiments. The genomics content may be, for example, translational genomics content. It may also be, for example, expressional genomics content (e.g., information about gene expression levels in various circumstances). System 200 can include server computer 210 and a database component 220. Server computer 210 can be a computer system such as computer system 100 of FIG. 1, for example.

Returning to FIG. 2, database component 220 is shown as a separate device from server computer 210. In various embodiments, database component 220 can be part of server computer 210. Database component 220 can also be shown directly connected to server computer 210. In various embodiments, database component 220 can be indirectly connected to server computer 210, such as through network 250. Database component 220 can include one or more physical devices. Database component 220 can include electronic and/or magnetic components, for example.

Server computer 210 is configured to receive genomic data (e.g., genomic variation data, etc.) and contributor data (e.g., translational genomics content, links to translational genomics content, gene expression data, etc.) from a contributor client device 240 and store the genomic data and contributor data in the database component 220. Server computer 210 can be configured to receive and send information from and to a contributor client device 240 through network 250, for example. In various embodiments, network 250 can include the Internet. A contributor client device 240 can include, but is not limited to, a computer, mobile phone, a tablet computer, a laboratory instrument, or any device capable of sending or receiving electronic information. Genomic data can include, but is not limited to, a genomic sequence, a logical association with a genomic sequence, a medically relevant variation and a relationship between the variation and a medical condition of a patient, gene expression data, etc. The medically relevant variation and the relationship can be described in published data or a proprietary study, for example.

Server computer 210 can be configured to determine an association between the genomic data and other contributor data stored in database component 220 and store the association in the database component 220. An association between the genomic data and contributor data stored in database component 220 can be found using a Web or network crawler, for example. In various embodiments, server computer 210 can be configured to determine an association between the genomic data and contributor data stored in the electronic database by searching at least one other server computer for information about the genomic data or contributor data. In this manner, the information in database component 220 may be self-generating and self-perpetuating, and it may grow both as a result of the direct submission of information by contributors and as a result of new links and associations that may be made as the server computer 210 searches for additional information associated with or publicly known to be related to information directly submitted by contributors.

Server computer 210 can be configured to receive a request for the contributor data from a user client device 260, retrieve the contributor data from electronic database 220, and send the contributor data to the client device 260. Server computer 210 can be configured to receive and send information from and to a user client device 260 through network 250, for example. A user client device 260 can include, but is not limited to, a computer, mobile phone, a tablet computer, a laboratory instrument, or any device capable of sending or receiving electronic information.

Finally, server computer 210 can be configured to calculate a compensation amount value for the contributor data and store the compensation amount value in a data file associated with the contributor. In various embodiments, the compensation amount value for the contributor data can be stored in database component 220.

As described above, the compensation value calculated for the contributor data can be proportional to the value or reliability of the information provided. The value of the contributor data can be established based on a number of factors, which may be used toward this calculation independently or collectively in various groups of two or more of them.

One factor on which the value of the contributor data can be based is the number of times a contributor data is accessed by users. In Web parlance, the number of hits a contributor data receives may be directly proportional to its value, for example. In various embodiments, server computer 210 can be configured to store the number of times the request for the contributor data is received and use the number of times the request for the contributor data is received to calculate the compensation amount value for the contributor data. The compensation amount value may be a function of the number of hits. For example, the compensation amount value may be directly proportional to the number of hits. More generally, the compensation amount value may be any function that increases, linearly or non-linearly, as the number of hits increases.

Another factor on which the value of contributor data can be based is the credibility of the contributor. For example, a contributor from a well-respected institution can be assumed to have more credibility than a contributor from a little known institution. In various embodiments, server computer 210 may be configured to assign a level of validation to the contributor data based on information (e.g., a number representing credibility) about a contributor provided in the contributor data or other contributors and use the level of validation to calculate the compensation amount value for the contributor data. The compensation amount value may be any function of the level of credibility of the contributor that yields a higher compensation amount for a higher level of credibility.

Another factor on which the value of a contribution can be based is the type of study used to generate the contributor data. For example, an institution wide or government run study can have more credibility than a study conducted by one person on his or her own. Further, an element of information validated through some finding or conclusion in a study or review conducted by an appropriate regulatory body (e.g., the FDA or its foreign equivalents) could have a high level of credibility. The type of the study may also be characterized by the specificity of any clinical correlations or the level of statistical significance of any conclusion it may contain (e.g., a p-value, type I and II statistical error levels, etc.), with higher levels of specificity and/or statistical significance yielding higher validation levels. Further, a study that has been approved or favorably recognized by an independent party may be deemed more reliable. For example, approval by an insurance company and/or payor of a study may bolster the level of validation of the study. In various embodiments, server computer 210 can be configured to assign a level of validation to the contributor data based on information about the type of study provided in the contributor data and use the level of validation to calculate the compensation amount value for the contributor data. The compensation amount value may be any function of the level of credibility of the study that yields a higher compensation amount for a higher level of credibility.

Another factor on which the value of a contributor data can be based is the frequency of citation of the contributor data in the literature or the prestige ranking of a journal that published the data. A published paper that is cited more frequently by other published papers can be more reliable, for example. In various embodiments, server computer 210 can be configured to assign a level of validation to the contributor data based on information about the frequency of citation of the contributor data provided in the contributor data and use the level of validation to calculate the compensation amount value for the contributor data. The compensation amount value may be any function of the frequency of citation that yields a higher compensation amount for more frequently cited information.

Another factor on which the value of a contributor data can be based is the age of the contribution. A published paper that is more recent can be more valuable because its authors likely were aware of prior studies and publications on the same topic. The authors of that paper could have considered and addressed any discrepancy between any new results relative to previously published results. In addition, a more recently published paper that summarizes and reviews older studies can be more valuable than the original studies because it implies that the information in those studies was further critically reviewed for validity. In various embodiments, server computer 210 can be configured to assign a level of validation to the contributor data based on information about the age of the contributor data provided in the contributor data and use the level of validation to calculate the compensation amount value for the contributor data.

Another factor on which the value of a contributor data can be based is the relationship of the contributor data to a scientific controversy. Controversy about a contribution can decrease its value, for example. In various embodiments, server computer 210 can be configured to assign a level of validation to the contributor data based on information about the relationship of the contributor data to a scientific controversy provided in the contributor data and use the level of validation to calculate the compensation amount value for the contributor data.

Another factor on which the value of a contribution can be based is the size or depth of the contribution. A larger set of information relating to sequence information can have greater value than just one piece of information, for example. For example, a element of information containing only a suspected association between a genomic variation and a medical condition could be less valuable than an equally credible element of information that also suggests an appropriate treatment corresponding that genomic variation and medical condition. In various embodiments, server computer 210 can be configured to assign a level of validation to the contributor data based on the size or depth of the contributor data and use the level of validation to calculate the compensation amount value for the contributor data.

Another factor on which the value of a contribution can be based is the feedback received from users of the contribution or from other sources. The feedback can include reviews of the contribution, for example, which may result from feedback of other users or from review of the contributed information by a regulatory body such as the FDA, for example. The type of feedback can include opinions by users. It may also include published conclusions by other scientists and/or regulatory government officers about the specificity of a clinical correlation or a level of statistical significance of a contributed piece of information. In various embodiments, server computer 210 can be further configured to receive feedback information from the user client device for the contributor data, assign a level of validation to the feedback information, and use the level of validation to calculate the compensation amount value for the contributor data.

Another factor on which the value of a contribution can be based is whether the contribution is proprietary or public information. Proprietary information can have a higher value than public information, for example. Patented information can also have a higher value than other types of information. In various embodiments, server computer 210 can be configured to assign a level of validation to the contributor data based on whether the contributor data is proprietary or public according to information provided in the contributor data and use the level of validation to calculate the compensation amount value for the contributor data.

As described above, the information contributed to the database can be binned into one or more categories based its value. Contributions may be ranked based on compensation amount, for example. In various embodiments, server computer 210 can be further configured to calculate a rank for the contributor data based on the compensation amount value and store the rank in the electronic database.

As described above, contributors can be given additional incentives to contribute to the database. For example, contributors can be given a reduced price for access to the database. In various embodiments, server computer 210 can be further configured to receive user data with the request and store the request and the user data in electronic database 220 as a charge for a user of the user client device. Server computer 210 can be configured to compare the user data to contributor data in electronic database 220 and reduce the amount of the charge if the user data matches any contributor data stored in electronic database 220. Also, the server computer 210 may be further configured to detect attempts by contributors are manipulating one or more of the above factors to increase the compensation they are entitled to for their contributions. For example, feedback by a contributor or entities associated with the contributor about the contributor's contributed information may be downplayed or ignored in determining that contributor's compensation. Alternatively, the compensation may be capped in various ways calculated to deter and render ineffective any such attempts at manipulating the system.

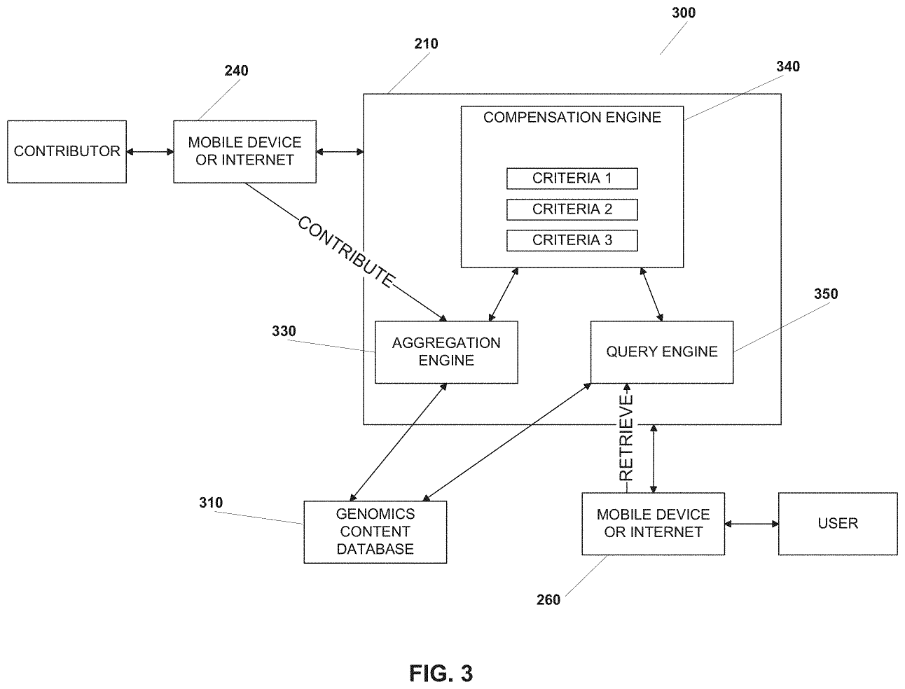

FIG. 3 is a schematic diagram showing a system 300 for aggregating and valuing genomics content. The genomics content may be, for example, translational genomics content or gene expression content. System 300 can include genomics content database 310 and a server computer 210. Server computer 210 includes an aggregation engine 330, a genomics content compensation engine 340 and a query engine 350. Contributor client device 240 can send genomics content to the aggregation engine 330, which is configured to establish an association between the genomics content and genomic variation data stored in the genomics content database 310. The genomics compensation engine 340 can be configured to calculate a compensation value for the genomics content based on a set of criteria (such as a level of validation for the genomics content). The query engine 350 can be configured to receive user requests (from user client device 260) for genomics content associated with a genomic variation, retrieve the genomics content associated with the genomic variation, and send the associated content to the user client device 260.

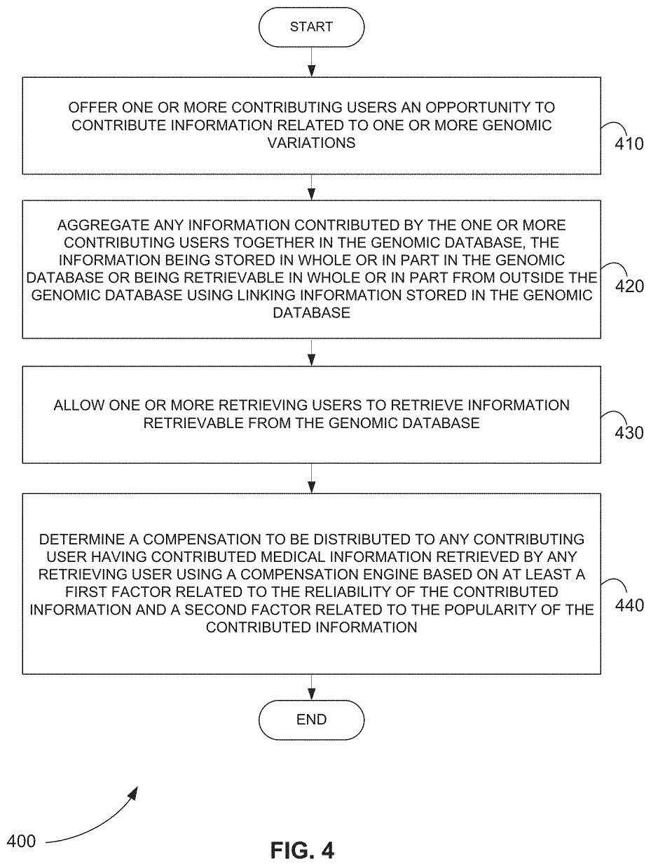

FIG. 4 is a flowchart showing a method 400 for generating a genomic database, in accordance with various embodiments.

In step 410 of method 400, one or more contributing users can be offered an opportunity to contribute information related to one or more genomic variations. The information may include, for example, information of a medical nature related to a patient, treatment response, etc.

In step 420, any information contributed by the one or more contributing users can be aggregated together in the genomic database. The information can be stored in whole or in part in the genomic database, for example. In various embodiments, the information can be retrieved in whole or in part from outside the genomic database using linking information stored in the genomic database.

In step 430, one or more retrieving users can be allowed to retrieve information retrievable from the genomic database.

In step 440, a compensation to be distributed to any contributing user having contributed information retrieved by any retrieving user can be determined using a compensation engine. The compensation can be based on at least a first factor related to the reliability of the contributed information of the contributing user having contributed the contributed information and a second factor related to the popularity of the contributed information.

In various embodiments, a computer program product can include a non-transitory and tangible computer-readable storage medium whose contents include a program with instructions being executed on a server computer so as to perform a method for generating a genomic database. This method can be performed by a system that includes one or more distinct software modules, for example.

While the present teachings are described in conjunction with various embodiments, it is not intended that the present teachings be limited to such embodiments. On the contrary, the present teachings encompass various alternatives, modifications, and equivalents, as will be appreciated by those of skill in the art. While various embodiments, have been described with respect to the use of genetic or genomic sequence information, it should be understood that, according to the present teachings, other types of biological information can be used instead of or in conjunction with the genetic or genomic sequence information.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

In a first aspect, a system for aggregating and valuing biological information can include at least one biological database; and a server computer in communication with the at least one biological database. The server can include an aggregation engine, a query engine, and a biological information compensation engine. The aggregation engine can be configured to, receive biological information from a contributor client device, and establish an association between the biological information and biological data stored in the biological database. The query engine can be configured to, receive a request for biological information associated with biological data from a user client device, retrieve the biological information associated with the biological data from the biological database, and provide the associated biological information to the user client device. The biological information compensation engine can be configured to determine a compensation value for the biological information based on at least one criterion related to a characteristic of the biological information. In various embodiments of the first aspect, the biological data can include a biological sequence.

In various embodiments of the first aspect, the association between the biological data and the biological information can comprise a correlation between one or more biological sequence variations and a medical condition of a patient. In particular embodiments, the biological information can comprise data from published or proprietary studies.

In various embodiments of the first aspect, the biological database can be stored in the server computer. In various embodiments of the first aspect, the aggregation engine, the query engine, and the biological information compensation engine can form a unified system or application.

In various embodiments of the first aspect, the aggregation engine that can be configured to determine the association between the biological data and the biological information by querying at least one other server computer for information relating to the biological data.

In various embodiments of the first aspect, the biological information compensation engine can be further configured to determine the number of times the biological information is retrieved and use the number of times the biological information is retrieved to determine the compensation amount value for the biological information. In various embodiments of the first aspect, the biological information compensation engine can be further configured to associate a validation level to a biological information contributor. In particular embodiments, the biological information compensation engine can be configured to utilize the validation level to determine the compensation amount value for biological information submitted by the biological information contributor.

In various embodiments of the first aspect, the biological information compensation engine can be configured to associate a validation level to the biological information based on whether the biological information is published data or proprietary data and use the validation level to determine the compensation amount value for the biological information.

In various embodiments of the first aspect, the biological information compensation engine can be configured to associate a validation level to the biological information based on a frequency of citation of the biological information provided in publications or other biological information and use the validation level to determine the compensation amount value for the biological information.

In various embodiments of the first aspect, the biological information compensation engine can be configured to associate a validation level to the biological information based on biological information age and use the validation level to determine the compensation amount value for the biological information.

In various embodiments of the first aspect, the biological information compensation engine can be configured to associate a validation level to the biological information based on a representative of scientific controversy value and use the validation level to determine the compensation amount value for the biological information.

In various embodiments of the first aspect, the biological information compensation engine can be configured to receive feedback information from the user client device for the biological information, associate a validation level to the feedback information, and use the validation level to determine the compensation amount value for the biological information.

In various embodiments of the first aspect, the biological information compensation engine can be configured to associate a validation level to the biological information based on whether the biological information is proprietary or public and use the validation level to determine the compensation amount value for the biological information.

In various embodiments of the first aspect, the biological information compensation engine can be configured to determine a rank for the biological information based on the compensation amount value and store the rank in the biological database.

In various embodiments of the first aspect, the query engine can be configured to receive user data with the request and store the request and the user data in the biological database. The user can be charged on the basis of the biological information provided. The user may not be charged for biological information which the user has contributed.

In various embodiments of the first aspect, the biological information compensation engine can be further configured to (1) detect an attempt by a contributor at increasing the compensation value by submitting information calculated to favorably impact the at least one criterion, and (2) downplay or ignore the submitted information calculated to favorably impact the at least one criterion so as to nullify the contributor's attempt.

In a second aspect, a method for generating a biological database can include offering one or more contributing users an opportunity to contribute information related to one or more biological variations, aggregating any information contributed by the one or more contributing users together in the biological database, the information being stored in whole or in part in the biological database or being retrievable in whole or in part from outside the biological database using linking information stored in the biological database, allowing one or more retrieving users to retrieve information retrievable from the biological database; and determining a compensation to be distributed to any contributing user having contributed information retrieved by any retrieving user using a compensation engine based on at least a first factor related to the reliability of the contributed information and a second factor related to the popularity of the contributed information.

In various embodiments of the second aspect, the first factor can include information about a reliability of the contributing user having contributed the contributed information. In various embodiments of the second aspect, the first factor can include information about the type of study upon which the contributed information is based. In various embodiments of the second aspect, the first factor can include information about the frequency of citation of the contributed information. In various embodiments of the second aspect, the first factor can include information about the age of the contributed information. In various embodiments of the second aspect, the first factor can include information about the relationship of the contributed information to a scientific controversy. In various embodiments of the second aspect, the first factor can include information about the size or depth of the contributed information. In various embodiments of the second aspect, the first factor can include whether the contributed information is proprietary or public.

In various embodiments of the second aspect, the method can further include (1) monitoring the contributed information to detect an attempt by a contributor at increasing the compensation to be distributed to that contributor by submitting information calculated to favorably impact one or more of the first and second factors, and (2) downplaying or ignoring any submitted information calculated to favorably impact one or more of the first and second factors so as to nullify the contributor's attempt.

In a third aspect, a computer program product can include a non-transitory and tangible computer-readable storage medium whose contents include a program with instructions being executed on a server computer so as to perform a method for generating a biological database. The method can include offering one or more contributing users an opportunity to contribute information related to one or more biological variations, aggregating any information contributed by the one or more contributing users together in a biological database, the information being stored in whole or in part in a biological database or being retrievable in whole or in part from outside the biological database using linking information stored in the biological database, allowing one or more retrieving users to retrieve information retrievable from the biological database, and determining a compensation to be distributed to any contributing user having contributed information retrieved by any retrieving user using a compensation engine based on at least a first factor related to the reliability of the contributed information and a second factor related to the popularity of the contributed information.

In various embodiments of the third aspect, the first factor can include information about a reliability of the contributing user having contributed the contributed information. In various embodiments of the third aspect, the first factor can include information about the type of study upon which the contributed information is based. In various embodiments of the third aspect, the first factor can include information about the frequency of citation of the contributed medical information. In various embodiments of the third aspect, the first factor can include information about the age of the contributed information. In various embodiments of the third aspect, the first factor can include information about the relationship of the contributed information to a scientific controversy. In various embodiments of the third aspect, the first factor can include information about the size or depth of the contributed information. In various embodiments of the third aspect, the first factor can include whether the contributed information is proprietary or public.

In various embodiments of the third aspect, the method can further include (1) detecting an attempt by a contributor at increasing the compensation to be distributed to that contributor by submitting information calculated to favorably impact one or more of the first and second factors, and (2) downplaying or ignoring the submitted information calculated to favorably impact one or more of the first and second factors so as to nullify the contributor's attempt.

In a fourth aspect, a method for generating a biological database can include aggregating information contributed by one or more contributing users together in a biological database, the information being stored in whole or in part in the biological database or being retrievable in whole or in part from outside the biological database using linking information stored in the biological database; and determining a compensation to be distributed to any contributing user having contributed information based on at least a first factor related to the reliability of the contributed information.

In various embodiments of the fourth aspect, determining the compensation can further include determining the compensation based on a second factor related to the popularity of the contributed information.

1.-20. (canceled)

21. A method for generating a biological database, comprising:
offering one or more contributing users an opportunity to contribute information related to one or more biological variations;
aggregating any information contributed by the one or more contributing users together in the biological database, the information being stored in whole or in part in the biological database or being retrievable in whole or in part from outside the biological database using linking information stored in the biological database;
allowing one or more retrieving users to retrieve information retrievable from the biological database; and
determining a compensation to be distributed to any contributing user having contributed information retrieved by any retrieving user using a compensation engine based on at least a first factor related to the reliability of the contributed information and a second factor related to the popularity of the contributed information.

22. The method of claim 21, wherein the first factor comprises information about a reliability of the contributing user having contributed the contributed information.

23. The method of claim 21, wherein the first factor comprises information about the type of study upon which the contributed information is based.

24. The method of claim 21, wherein the first factor comprises information about the frequency of citation of the contributed information.

25. The method of claim 21, wherein the first factor comprises information about the age of the contributed information.

26. The method of claim 21, wherein the first factor comprises information about the relationship of the contributed information to a scientific controversy.

27. The method of claim 21, wherein the first factor comprises information about the size or depth of the contributed information.

28. The method of claim 21, wherein the first factor comprises whether the contributed information is proprietary or public.

29. The method of claim 21, further comprising (1) monitoring the contributed information to detect an attempt by a contributor at increasing the compensation to be distributed to that contributor by submitting information calculated to favorably impact one or more of the first and second factors, and (2) downplaying or ignoring any submitted information calculated to favorably impact one or more of the first and second factors so as to nullify the contributor's attempt.

30. A computer program product, comprising a non-transitory and tangible computer-radable storage medium whose contents include a program with instructions being executed on a server computer so as to perform a method for generating a biological database, the method comprising:
offering one or more contributing users an opportunity to contribute information related to one or more biological variations;
aggregating any information contributed by the one or more contributing users together in the biological database, the information being stored in whole or in part in the biological database or being retrievable in whole or in part from outside the biological database using linking information stored in the biological database;
allowing one or more retrieving users to retrieve information retrievable from the biological database; and
determining a compensation to be distributed to any contributing user having contributed information retrieved by any retrieving user using a compensation engine based on at least a first factor related to the reliability of the contributed information and a second factor related to the popularity of the contributed information.

31. The computer program product of claim 30, wherein the first factor comprises information about a reliability of the contributing user having contributed the contributed information.

32. The computer program product of claim 30, wherein the first factor comprises information about the type of study upon which the contributed information is based.

33. The computer program product of claim 30, wherein the first factor comprises information about the frequency of citation of the contributed information.

34. The computer program product of claim 30, wherein the first factor comprises information about the age of the contributed information.

35. The computer program product of claim 30, wherein the first factor comprises information about the relationship of the contributed information to a scientific controversy.

36. The computer program product of claim 30, wherein the first factor comprises information about the size or depth of the contributed information.

37. The computer program product of claim 30, wherein the first factor comprises whether the contributed information is proprietary or public.

38. The computer program product of claim 30, the method further comprising (1) detecting an attempt by a contributor at increasing the compensation to be distributed to that contributor by submitting information calculated to favorably impact one or more of the first and second factors, and (2) downplaying or ignoring the submitted information calculated to favorably impact one or more of the first and second factors so as to nullify the contributor's attempt.

39. A method for generating a biological database, comprising:
aggregating information contributed by one or more contributing users together in the biological database, the information being stored in whole or in part in the biological database or being retrievable in whole or in part from outside the biological database using linking information stored in the biological database; and
determining a compensation to be distributed to any contributing user having contributed information based on at least a first factor related to the reliability of the contributed information.

40. The method of claim 39, wherein determining the compensation further includes determining the compensation based on a second factor related to the popularity of the contributed information.
